(19) United States
(12) Patent Application Publication
(10) Pub. No.: US 2007/0212432 A1
(43) Pub. Date: Sep. 13, 2007

(54) Herbal Composition

(75) Inventor: Karina Anna Hilterman, Hawthorn East (AU)

Correspondence Address: NEEDLE & ROSENBERG, P.C., SUITE 1000, 999 PEACHTREE STREET, ATLANTA, GA, 30309-3915, US

(73) Assignee: LAVENDER HILL PROJESTS PROJECTS PTY LTD, HAWTHORN EAST VICTORIA Australia (AU)

(21) Appl. No.: 11/578,532

(22) PCT Filed: Apr. 14, 2004
(86) PCT No.: PCT/AU04/00489

§ 371 Date: Oct. 13, 2006

(30) Foreign Application Priority Data
Apr. 16, 2003 (NZ) 525425

(51) Int. Cl. A61K 36/00; A61K 36/28; A61K 36/48

(52) U.S. Cl. 424/737; 424/725; 424/757

(57) ABSTRACT

The invention relates to a herbal composition comprising extracts from Thuja, Bupleurum, Echinacea, Calendula, Licorice and Hypercum; formulations containing the herbal composition; and use of the herbal composition for alleviating, treating and/or preventing sypmtoms associated with or caused by viral infections

The invention relates to a herbal composition. Specifically, the invention relates to a herbal composition suitable for alleviating, treating and/or preventing symptoms associated with or caused by viral infections.

### BACKGROUND

The use of plants and plant extracts in the treatment of disease and illness has occurred for centuries. Many of the medicines that are used today originate from early herbal remedies and formulations. For example, morphine and codeine are derived from opium poppies, while taxol, which is used in the treatment of breast cancer, is derived originally from the Pacific Yew tree.

Viruses are super molecular complexes of nucleic acids, either DNA or RNA, encapsulated in a protein coat. Viruses may infect animal cells and plant cells. Once a virus gains access to the specific host cell, it can alter or manipulate the function of the host cell producing undesirable side effects.

Viruses of the Herpesviridae family of viruses such as Herpes Simplex Virus (HSV), Epstein-Barr Virus (EBV) and Cytomegalo Virus (CMV) are amongst the most common viruses affecting adults and children. Another common family of viruses is the Papovaviridae family of viruses, which includes the Human Papilloma Virus (HPV). Coxsackie B virus is also a common virus, most prominently affecting children.

Herpes viruses (Herpesviridae) are another class of virus common in the general population and include Herpes Simplex Virus (HSV), which causes irritable skin lesions, Epstein-Barr Virus (EBV), which causes infectious mononucleosis, and Cytomegalo Virus (CMV), which causes cytomegalo virus inclusion disease.

There are two different strains of HSV. Herpes simplex virus type 1 (HSV-1) is usually associated with infections of the lips, mouth, and face. It is the most common herpes simplex virus among the general population and is usually acquired in childhood. HSV-1 often causes lesions inside the mouth such as cold sores. Herpes simplex virus 2 (HSV-2) is sexually transmitted and is usually associated with genital ulcers or sores.

EBV is most present in adolescent populations in developed countries. Transmission appears to be exposure to EPV-contaminated saliva. The virus undergoes a replicated cycle in the oropharyngeal epithelium and then invades the blood by infecting B cells. The infection manifests itself by fever, sore throat, and the appearance in the blood of atypical lymphocytes. For EBV, the treatments include bed rest and taking analgesic (aspirin) to relieve the fever and headaches.

CMV is primarily a sexually transmitted disease and manifests itself in mononucleosis-like symptoms, which include fever and body aches. In some cases, the virus may be without symptoms at all. People with HIV seem to be preferentially infected via the eye in the form of CMV retinitis.

Many different types of Human Papilloma Virus (HPV) cause warts, which are a common epithelial tumor. HPV infects skin cells epithelial keratinocytes. The viruses replicate in skin cells, causing a variety of external growths. The usual mode of transmission of the virus is by skin to skin inoculation, although it is not well understood how the virus penetrates and infects cells.

There is often a wide variation in time between infection and manifestation of visible warts: with common warts from several days to several weeks. Genital warts may not appear until more than a year after infection. It is believed that some resistance to the manifestation may be due to cell mediating immune responses.

Current treatments for symptoms of HPV, such as warts, include burning or freezing the infected area of skin. In some instances the infected area may be surgically removed. All these treatments can be painful and may not prevent warts from returning. Generally the current treatment can broadly be defined as topical and symptomatic.

The current orthodox methods for treating symptoms of viral infections are few and of limited benefit. Therefore, there is a need to provide an improved treatment for symptoms of viral infections or to at least provide the public with a useful, alternative choice of treatment.

### SUMMARY

According to a first aspect of the invention, there is provided a herbal composition comprising extracts from:
Thuja;
Bupleurum;
Echinacea;
Calendula;
Licorice; and
Hypericum.

The herbal composition may further comprise extracts of one or more of Manuka, Lycium, Burdock, Cats Claw, Poke root, Ginger, Thyme, Barberry, Golden Seal and Rehmannia, Schisandra, and St Mary's Thistle.

A second aspect of the invention provides a method of alleviating, treating and/or preventing symptoms of skin lesions, sores, cold sores, blisters, warts, lumps, bumps, pimples, rashes and ulcers associated with or caused by a viral infection which comprises administering a therapeutically effective amount of the herbal composition as defined above to a subject in need thereof.

A third aspect of the invention provides use of the herbal composition as defined above for the manufacture of a medicament to alleviate, treat and/or prevent symptoms of skin lesions, sores, cold sores, blisters, warts, lumps, bumps, pimples, rashes and ulcers associated with or caused by a viral infection.

A fourth aspect of the invention provides use of the herbal composition as defined above to alleviate, treat and/or prevent symptoms of skin lesions, sores, cold sores, blisters, warts, lumps, bumps, pimples, rashes and ulcers associated with or caused by a viral infection.

A fifth aspect of the invention provides the herbal composition as defined above for use in alleviating, treating and/or preventing symptoms of skin lesions, sores, cold sores, blisters, warts, lumps, bumps, pimples, rashes and ulcers associated with or caused by a viral infection.

A sixth aspect of the invention provides an agent comprising the herbal composition as defined above for alleviating, treating and/or preventing symptoms of skin lesions, sores, cold sores, blisters, warts, lumps, bumps, pimples, rashes and ulcers associated with or caused by a viral infection.

The viral infections include, but are not limited to, the Herpesviridae family of viruses such as Herpes Simplex Virus (HSV), Epstein-Barr Virus (EBV) and Cytomegalo Virus (CMV); the Papovaviridae family of viruses such as Human Papilloma Virus (HPV); and the Coxsackie B virus.

The herbal composition may conveniently be administered together with one or more carriers. Thus a seventh aspect of the invention provides the herbal composition as defined above together with one or more carriers.

The herbal composition is suitably administered orally and/or topically.

### DETAILED DESCRIPTION

In the subject specification, except where the context requires otherwise due to express language or necessary implication, the words “comprise” or variations such as “comprises” or “comprising” are used in an inclusive sense, i.e. to specify the presence of the stated features but not to preclude the presence or addition of further features in various embodiments of the invention.

It must be noted that, as used in the subject specification, the singular forms “a”, “an” and “the” include plural aspects unless the context clearly dictates otherwise. Thus, for example, reference to “an extract” includes a single extract, as well as two or more extract; and so forth.

The term “extract” as used herein should be taken in the broadest possible sense. “Extracts” may include tinctures, fluid extracts or solid extracts, for example.

In a preferred form of the invention, the extracts are tinctures. Tinctures may be formed from water-based infusions and decoctions. Alternative bases for the extract may include acetracts (to form a vinegar extract), glycetracts (to form a glycerine extract), mels (to form a honey extract), oxymels (honey and vinegar), or syrups (to form a sugar extract).

Tinctures are prepared by methods well known in the art. Briefly, however, a fresh plant tincture is made by first obtaining a herb dried by air as known in the art. The dried herbs are then further dried in an oven at a temperature of between 60° C. and 70° C. The oven dried herbs are then suspended in a solution in a storage container. In a preferred form, the solution is a mixture of alcohol and water. To make up a 1:5 tincture, 75 gms of oven dried herb is suspended in 375 ml of the solution (weight of herb×5 to get 1:5 ratio) in a storage container. The storage container is allowed to stand for about 6 weeks and is shaken periodically. The mixture is then filtered and the solution withdrawn provides the tincture used.

Other methods of extracting herbs are well known in the field. For example, cold percolation techniques may be used to prepare tinctures.

The tinctures may also be purchased from herbal suppliers such as MediHerb Pty Ltd, 124 McEvoy Street, Warwick, Queensland 4370, Australia in a ready-to-use formulation.

The extracts may be prepared from any part of the herb plant such as, for example, foliage, leaves, the root, flowers, bark, stems or rhisome, seeds, and fruit. However, particular parts of the herb plant are usually used to prepare the extracts.

A Thuja extract is usually made from the foliage. The herbal composition preferably comprises from about 0.9% to about 55% of Thuja extract, and more preferably about 18% of Thuja extract.

A Bupleurum extract is usually made from the root. The herbal composition preferably comprises from about 0.9% to about 72% of Bupleurum extract, and more preferably about 18% of Bupleurum extract.

An Echinacea extract is usually made from the root or the whole plant of the herb. The herbal composition preferably comprises from about 0.9% to about 55% of Echinacea extract, and more preferably about 23% of Echinacea extract.

A Calendula extract is usually made from the flowers. The herbal composition preferably comprises from about 0.9% to about 36% of Calendula extract, and more preferably about 9% of Calendula extract.

A Licorice extract is usually made from the root. The herbal composition preferably comprises from about 0.9% to about 36% of Licorice extract, and more preferably about 14% of Licorice extract.

A Hypericum extract is usually made from the flowers. The herbal composition preferably comprises from about 1.5% to about 55% of Hypericum extract, and more preferably about 18% of Hypericum extract.

The herbal composition may include one or more different kinds of extracts. For example, the different herbs may be processed to form extracts by different means. Similarly, the herbal composition may comprise a mixture of two or more types of extracts of a particular herb.

Table 1 illustrates a herbal composition of a preferred embodiment of the invention.

TABLE 1

| Common | | | | | |
|---|---|---|---|---|---|
| Name | Approved Name | Herb Part | Preparation | Volume | |
| Thuja | Thuja occidentalis | Foliage | 1:5 | 1-60 | ml |
| Bupleurum | Bupleurum falcatum | Root | 1:2 | 6-80 | ml |
| Echinacea | Echinacea purpurea | Root | 1:2 | 10-60 | ml |
| Calendula | Calendula | Flower | 1:2 | 1-40 | ml |
| | officinalis | | | | |
| Licorice | Glycyrrhiza glabra | Root | 1:1 | 1-40 | ml |
| Hypericum | Hypericum | Flower | 1:2 | 2-60 | ml |
| | perforatum | | | | |

In addition to the above-mentioned herb extracts, the herbal composition may further comprise one or more additional herb extracts, which may also assist in alleviating, treating and/or preventing symptoms of skin lesions, sores, cold sores, blisters, warts, lumps, bumps, pimples, rashes and ulcers associated with or caused by viral infections. Examples include herb extracts having anti-viral properties such as Manuka, Thyme and Barberry, or properties that stimulate the immune system such as Lycium, Burdock, Cats Claw, Poke root, Ginger, Golden Seal, Rehmannia, Schisandra, and St Mary's Thistle.

Manuka (i.e. Leptospermum scoparium) extract is usually made the leaves of the plant.

A Lycium (i.e. Lycium barbarum) extract is usually made from the fruit.

A Burdock (i.e. Arctium lappa) extract is usually made from the root.

R Cats Claw (i.e. Uncaria tormentosa) extract is usually made from inner bark of the vine.

A Poke root (i.e. Phytolacca decandra) extract is usually made from the root.

A Ginger (i.e. Zingerber officinale) extract is usually made from the root or rhizome.

A Thyme (i.e. Thymus spp) is usually made from the leaves of the plant.

A Barberry (i.e. Berberis vulgaris) extract is usually made from the inner bark of the plant.

A Golden Seal (i.e. Hydrastis canadensis) extract is usually made from the root or rhizome.

A Rehmannia (i.e. Rehmannia glutinosa) extract is usually made from the root.

A Schisandra (i.e. Schisandra chinensis) extract is usually made from the fruit.

A St Mary's Thistle (e.g. Silybum marianum) extract is usually made from the seed.

The herbal composition may comprise from about 0.1% to about 30%, more preferably from about 0.1% to about 10%, of each of the above-mentioned additional herb extracts.

The viral infections include, but are not limited to, the Herpesviridae family of viruses such as Herpes Simplex Virus (HSV), Epstein-Barr Virus (EBV) and Cytomegalo Virus (CMV); the Papovaviridae family of viruses such as Human Papilloma Virus (HPV); and the Coxsackie B virus.

Not wishing to be bound by a particular theory or hypothesis the inventor believes that the surprising and unexpected effects seen with the herbal composition results from synergism between the individual components.

The term “subject” as used herein refers to any animal having symptoms associated with or caused by viral infections, which requires treatment with the herbal composition. The subject may be an animal, such as a mammal, preferably a human, or may be a non-human primate or non-primates such as used in animal model testing. While it is particularly contemplated that the herbal composition is suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates.

Generally, the terms “treating”, “treatment” and the like are used herein to mean affecting a subject, tissue or cell to obtain a desired pharmacological and/or physiological effect. The effect may be prophylactic in terms of completely or partially preventing one or more symptoms of skin lesions, sores, cold sores, blisters, warts, lumps, bumps, pimples, rashes and ulcers associated with or caused by a viral infection, and/or may be therapeutic in terms of a partial or complete cure of one or more symptoms of skin lesions, sores, cold sores, blisters, warts, lumps, bumps, pimples, rashes and ulcers associated with or caused by a viral infection. “Treating” as used herein covers any alleviation, treatment, or prevention of one or more symptoms of skin lesions, sores, cold sore, blisters, warts, lumps, bumps, pimples, rashes and ulcers associated with or caused by a viral infection in an animal such as a mammal, more particularly a human, and includes:
(a) preventing one or more of the symptoms referred to above from occurring in a subject that may be predisposed to such symptoms, but has not yet been diagnosed as having the viral infection;
(b) inhibiting one or more of the symptoms referred to above, i.e. arresting development of one or more of the symptoms referred to above; or
(c) alleviating, relieving or ameliorating the effects, i.e. cause regression, of one or more of the symptoms referred to above.

As used herein, the term “effective amount” means an amount of the herbal composition to alleviate, treat and/or prevent symptoms of skin lesions, sores, cold sores, blisters, warts, lumps, bumps, pimples, rashes and ulcers associated with or caused by a viral infection in order to yield a desired therapeutic response. For example, to overcome or alleviate the effects of one or more of those symptoms.

The term “therapeutically effective amount” means an amount of the herbal composition to yield a desired therapeutic response. For example, alleviating, treating and/or preventing the symptoms of skin lesions, sores, cold sores, blisters, warts, lumps, bumps, pimples, rashes and ulcers associated with or caused by a viral infection.

The herbal composition may conveniently be administered together with one or more carriers. Carriers include substances that are useful in preparing a formulation comprising the herbal composition, may be in co-administration with the composition while allowing the individual components to perform their intended function, and are generally safe, non-toxic and are neither biologically or otherwise undesirable. In addition, carriers will include those suitable for veterinarian use as well as human use. Examples of carriers include dispersing agents, suspending agents, emulsifying agents, stabilising agents, wetting agents, binding agents, lubricants, disintegrants, solvents, media, delay agents, fillers, aqueous and oily bases, non-aqueous vehicles, i.e. edible oils, and the like.

In addition, the herbal composition may contain preserving agents, sweetening agents, colouring agents, flavouring agents, thickening and/or gelling agents, buffers and the like. Those skilled in the art will be able to identify further additives that may be desirable for cosmetic reasons, palatability, or shelf life, for example.

As it will be appreciated by those skilled in the art to which the invention relates, the herbal composition may be converted into customary formulations. Examples of formulations include, but are not limited to, solutions, emulsions, suspensions, powders, granules, natural and synthetic materials impregnated with the individual components of the herbal composition, pills, capsules, tablets, cachets, pastilles, lozenges, bolus, electuary, pastes, ointments, creams, plasters, washes, lotions, transdermal patches, enemas, suppositories, pessaries, sprays (atomiser, or aerosol) mouthwashes, syrups, and/or elixirs. Preferably, the formulation is in the form of a solution or a cream. The inventor notes that in the case of warts (HPV infection) such formulations (i.e. oral solutions and topical creams) are particularly useful.

The choice of carriers and/or additives may be dictated to some extent by the intended dosage form of the herbal composition and the mode of administration of the herbal composition.

Formulations comprising the herbal composition may be produced by a number of techniques standard in the art, for example, by mixing the herbal extracts with one or more carriers and/or additives.

Formulations suitable for oral administration may conveniently be presented in discrete units such as capsules, cachets or tablets each containing a predetermined amount of the herbal composition; as a powder or granules; as a solution, a suspension or as an emulsion. The herbal composition may also be presented as a bolus, electuary or paste. Tablets and capsules for oral administration may contain conventional carriers such as binding agents, fillers, lubricants, disintegrants, or wetting agents. The tablets may be coated according to methods well known in the art. Oral liquid preparations may for example be in the form of aqueous or oily suspensions, emulsions, syrups or elixirs, or may be presented as a dry product, such as a powder or granules, for constitution with water or another suitable vehicle, e.g. orange juice, before use. Suitable carriers for such liquid preparations may include, for example, ethyl alcohol, vinegars, glycerine, honey, sugar, or water. Such liquid preparations may also contain conventional additives such as suspending agents, emulsifying agents, non-aqueous vehicles, which may include edible oils, or preservatives.

For topical administration to the skin, the herbal composition may be formulated as ointments, creams, plasters, washes, lotions, or as a transdermal patch. Ointments and creams may, for example, be formulated with an aqueous or oily base with the addition of suitable thickening and/or gelling agents. The Calendula present in the herbal composition may also be in the form of an oil infused with Calendula flower. Additionally, an essential oil of Thyme may be present. Lotions and washes may be formulated with an aqueous or oily base, and will in general also contain one or more emulsifying agents, stabilising agents, dispersing agents, suspending agents, thickening agents, or colouring agents.

Formulations suitable for topical administration in the mouth include lozenges comprising the herbal composition and may have a flavoured base such as sucrose and gum acacia or gum tragacanth; pastilles comprising the herbal composition in an inert base such as gelatin or sucrose and gam acacia; and mouthwash in a suitable liquid carrier.

In use, the herbal composition would be administered to a subject as needed, as desired, or as advised by a medical practitioner, pharmacist, attendant physician, medical herbalist, naturopath, or veterinarian. The selection of the herb extracts may also be made on the specific needs of the subject.

As will be appreciated, the dose administered, the period of administration, and the general administration regime may differ between subjects depending on variables such as their tolerance to certain active ingredients, weight, metabolism, the mode of administration chosen, the severity of the symptoms, and the age and/or general health of the subject. In general, however, a suitable dose of an oral formulation is preferably in the range of from about 1 ml to about 50 ml, at least once daily, more preferably from about 6 ml to about 10 ml, at least twice daily, most preferably about 8 ml at least twice daily. Oral treatment may also be supplemented with a topical application.

Treatment is preferably commenced before or at the time symptoms of skin lesions, sores, cold sores, blisters, warts, lumps, bumps, pimples, rashes and ulcers associated with or caused by a viral infection develop and preferably continues until such symptoms are no longer present.

The invention will now be further elucidated by reference to the following non-limiting example.

### EXAMPLE 1

Study Design

The study was designed for 40 participants.

Participants in the study were selected from the public.

Respondents with any pre-existing health condition that may have been adversely affected by the herbal composition were excluded from the trial. These conditions included elevated blood pressure, kidney insufficiency, pregnancy, lactation, and epilepsy.

The respondents had a wide range of “skin lumps” that they called warts, including a range of keratoses and some basal cell carcinomas.

After screening the prospective participants, 40 participants were chosen who met the following criteria:
ages of 18 to 70,
having benign warts verified by a medical practitioner; and
no serious pre-existing medical condition.

Each of the 40 participants received an individual consultation, with the principal researcher taking comprehensive medical history notes, including the history of their warts and any previous treatments for them. Participants had their blood pressure measured and also completed a medical history form.

Participants had their warts photographed, identified by their code number placed next to each wart. The scale was determined by placing a ruler in the photograph.

Control Group

Of the 40 participants, 20 participants were randomly selected to form the control group.

The control group had no treatment and at the end of the 12-week trial period, each participant in this group had their warts re-photographed.

Treatment Group

The treatment group, which consisted of the remaining 20 participants, took a herbal composition prepared as an oral formulation comprising:
20 ml of Thuja extract;
20 ml of Bupleurum extract;
25 ml of Echinacea purpurea extract;
10 ml of Calendula extract;
15 ml of Licorice extract; and
20 ml of Hypericum extract.

The extracts were sourced from MediHerb Pty Ltd of 124 McEvoy Street, Warwick, Queensland 4370, Australia. MediHerb combined the extracts to form the herbal composition. The herbal composition was prepared using a preferred extraction and composition technique (i.e. cold percolation technique).

The participants in the treatment group were required to take 8 ml of the oral herbal formulations twice daily, with food.

Each participant in the treatment group was also asked to fill in a compliance booklet, with a tick for each dose taken. This data was used to measure the degree of compliance by the participant.

At the end of the 12-week treatment period, the participants in the treatment group, as with the control group, were re-photographed.

Participant Withdrawals

By the end of the 12-week treatment period, there were two withdrawals from the treatment group: one woman became pregnant and the other was non-compliant. Therefore the relevant data for these two participants were not collected and are not included in the results. One participant withdrew from the control group.

Results

The results were measured by a visual comparison of the photographs before and after treatment.

Four categories were identified:

No Change
there was no apparent change in the warts (no patient had an increase in wart numbers or size);

Partially Cleared
up to 50% of the warts had been reduced or cleared;

Almost Cleared
more than 50% of the warts had been reduced or cleared; and

Totally Cleared
all sign of warts had disappeared.

The results were as follows:

| | Number of Participants | | | | |
|---|---|---|---|---|---|
| | | | | | Total |
| | | Partially | Almost | Totally | Completed |
| | No Change | Cleared | Cleared | Cleared | Study |
| Treatment | 5 | 6 | 2 | 5 | 18 |
| Control | 18 | 1 | 0 | 0 | 19 |

It is to be noted that the one participant in the control group who had any sign of change had been given a medical procedure which helped his body's ability to function better, which may have resulted in the spontaneous disappearance of most of his warts.

Adverse Reactions

No adverse reactions or effects were reported.

Statistical Significance of the Results

There was strong evidence of a difference in wart change between the two groups.

Statistical analysis showed that the probability that the differences seen between the two groups could have occurred if the effect of the treatment/no treatment was actually the same was 0.0007—i.e., there is only a chance of 7 in 10,000 that the effect of the treatment is actually the same (p=0.0007). This illustrates that the herbal composition of the present invention appears to show a positive effect for treatment. A p factor this low shows that it works.

The smaller the p value the more unlikely it is that the effect of the treatment is the same, meaning the more likely that the effect is real.

It is usually accepted that you need to have a p value of about 0.05 or smaller to demonstrate that there is a real difference between the treatments. The result of this trial is almost two orders of magnitude better than the basic p value of 0.05 and so there is high level of confidence in the effectiveness of the treatment.

Wherein the foregoing description reference has been made to specific component or integers of the invention which known equivalents then such equivalents are herein incorporated as if individually set forth.

Although this invention has been described by example and with reference to possible embodiment thereof, it is to be understood that modifications or improvements may be made thereto without departing from the scope of the invention.

1-37. (canceled)

38. A herbal composition comprising extracts from:
Thuja;
Bupleurum;
Echinacea;
Calendula;
Licorice; and
Hypericum.

39. The herbal composition according to claim 38, wherein the herbal composition comprises from about 0.9% to about 55% of Thuja extract.

40. The herbal composition according to claim 38, wherein the herbal composition comprises from about 0.9% to about 72% of Bupleurum extract.

41. The herbal composition according to claim 38, wherein the herbal composition comprises from about 0.9% to about 55% of Echinacea extract.

42. The herbal composition according to claim 38, wherein the herbal composition comprises from about 0.9% to about 36% of Calendula extract.

43. The herbal composition according to claim 38, wherein the herbal composition comprises from about 0.9% to about 36% of Licorice extract.

44. The herbal composition according to claim 38, wherein the herbal composition comprises from about 1.5% to about 55% of Hypericum extract.

45. The herbal composition according to claim 38, wherein the herbal composition comprises about 18% of Thuja extract.

46. The herbal composition according to claim 38, wherein the herbal composition comprises about 18% of Bupleurum extract.

47. The herbal composition according to claim 38, wherein the herbal composition comprises about 23% of Echinacea extract.

48. The herbal composition according to claim 38, wherein the herbal composition comprises about 9% of Calendula extract.

49. The herbal composition according to claim 38, wherein the herbal composition comprises about 14% of Licorice extract.

50. The herbal composition according to claim 38, wherein the herbal composition comprises about 18% of Hypericum extract.

51. The herbal composition according to claim 38, wherein the herbal composition further comprises extracts of one or more of Manuka, Lycium, Burdock, Cats Claw, Poke root, Ginger, Thyme, Barberry, Golden Seal and Rehmannia, Schisandra, and St Mary's Thistle.

52. The herbal composition according to claim 38, wherein the herbal composition further comprises from about 0.1% to about 30% of extracts of one or more of Manuka, Lycium, Burdock, Cats Claw, Poke root, Ginger, Thyme, Barberry, Golden Seal and Rehmannia, Schisandra, and St Mary's Thistle.

53. The herbal composition according to claim 38, wherein the herbal composition further comprises from about 0.1% to about 10% of extracts of one or more of Manuka, Lycium, Burdock, Cats Claw, Poke root, Ginger, Thyme, Barberry, Golden Seal and Rehmannia, Schisandra, and St Mary's Thistle.

54. The herbal composition according to claim 38, wherein one or more of the extracts is in the form of a tincture, a fluid extract or solid extract, or a mixture thereof.

55. The herbal composition according to claim 38, wherein one or more of the extracts is in the form of a tincture.

56. The herbal composition according to claim 55, wherein the tincture is formed from water-based infusions and decoctions.

57. The herbal composition according to claim 38, wherein the base for one or more of the extracts is an acetract to form a vinegar extract, a glycetract to form a glycerine extract, a mel to form a honey extract, an oxymel to form a honey and vinegar extract, or a syrup to form a sugar extract.

58. The herbal composition according to claim 38, wherein the composition is in an effective amount to alleviate, treat and/or prevent symptoms of skin lesions, sores, cold sores, blisters, warts, lumps, bumps, pimples, rashes and ulcers associated with or caused by a viral infection.

59. A herbal formulation comprising the herbal composition according to claim 38 and one or more carriers.

60. The herbal formulation according to claim 59, wherein the herbal formulation is in the form of an oral formulation.

61. The herbal formulation according to claim 60, wherein the oral formulation is a solution.

62. The herbal formulation according to claim 59, wherein the herbal formulation is in the form of a topical formulation.

63. The herbal formulation according to claim 62, wherein the topical formulation is a cream.

64. A method of alleviating, treating and/or preventing symptoms of skin lesions, sores, cold sores, blisters, warts, lumps, bumps, pimples, rashes and ulcers associated with or caused by a viral infection comprising administering a therapeutically effective amount of the herbal composition according to claim 38 to a subject in need thereof.

65. The method according to claim 64, wherein the viral infection is one or more of the Herpes Simplex Virus (HSV), the Epstein-Barr Virus (EBV), the Cytomegalo Virus (CMV), the Human Papilloma Virus (HPV), or the Coxsackie B virus.

66. The method according to claim 64, wherein the subject is an animal, mammal or human.

67. The method according to claim 64, wherein the herbal composition is in the form of an oral formulation.

68. The method according to claim 67, wherein from about 1 mL to about 50 mL of the oral formulation is administered at least once daily.

69. The method according to claim 67, wherein from about 6 mL to about 10 mL of the oral formulation is administered at least twice daily.

70. The method according to claim 67, wherein about 8 mL of the oral formulation is administered at least twice daily.

71. The method according to claim 64, wherein the herbal composition is administered in the form of an oral formulation and as a topical formulation.
